(19) United States
(12) Patent Application Publication
(10) Pub. No.: US 2024/0368579 A1
(43) Pub. Date: Nov. 7, 2024

(54) TARGETED NANODROPLET AND MICROBUBBLE COMPOSITIONS AND METHODS FOR ENRICHMENT, LYSIS, AND EXTRACTION OF MICROBIAL CELLS

(71) Applicants: Triangle Biotechnology, Inc., Chapel Hill, NC (US); The University of North Carolina at Chapel Hill, Chapel Hill, NC (US)

(72) Inventors: Sandeep Kasoji, Durham, NC (US); David Allison, Hillsborough, NC (US); Paul Dayton, Carrboro, NC (US); Jim Tsuruta, Chapel Hill, NC (US)

(21) Appl. No.: 18/689,948

(22) PCT Filed: Sep. 7, 2022
(86) PCT No.: PCT/US2022/076017

§ 371 Date: Mar. 7, 2024

Related U.S. Application Data
(60) Provisional application No. 63/241,214, filed on Sep. 7, 2021.

(51) Int. Cl. C12N 15/10; C12N 1/06

(52) U.S. Cl. CPC C12N 15/1006; C12N 1/066

(57) ABSTRACT

This invention relates to nanodroplet and microbubble particles comprising targeting ligands that bind to a cell of interest. The invention further relates to methods of using the targeted particles for lysis and extraction of cells of interest, such as microbial cells. The invention further relates to methods of using the targeted particles for enrichment of cells of interest, such as microbial cells.

### STATEMENT OF PRIORITY

This application claims the benefit of U.S. Provisional Application Ser. No. 63/241,214, filed Sep. 7, 2021, the entire contents of which are incorporated by reference herein.

### STATEMENT OF GOVERNMENT SUPPORT

This invention was made with government support under Grant Numbers CA236177 and CA232902 awarded by the National Institutes of Health. The government has certain rights in the invention.

### FIELD OF THE INVENTION

This invention relates to nanodroplet and microbubble particles comprising targeting ligands that bind to a cell of interest. The invention further relates to methods of using the targeted particles for lysis and extraction of cells of interest, such as microbial cells. The invention further relates to methods of using the targeted particles for enrichment of cells of interest, such as microbial cells.

### BACKGROUND OF THE INVENTION

The most effective technique for lysing resilient cell walls of microbial cells is bead-beating. However, drawbacks include poor consistency, biohazard risks related to aerosolization, complex and time-consuming workflows, and sample loss, thereby making it impractical for many infectious disease applications. See, e.g., Santaus T M, Li S, Saha L, et al. “A comparison of Lyse-It to other cellular sample preparation, bacterial lysing, and DNA fragmentation technologies”, PLoS One, 2019; 14 (7):e0220102; Jofuku K D, Schipper R D, Goldberg R B. “A frameshift mutation prevents Kunitz trypsin inhibitor mRNA accumulation in soybean embryos”, Plant Cell, 1989; 1 (5):567. Furthermore, studies show bead-beating may over-shear and produce cell debris and damage DNA, inhibiting purification steps and downstream PCR amplification for targeted Next Generation Sequencing (tNGS). Sonication is a promising alternative for lysis of resilient microbes, however the current gold-standard sonication technology, requires high-power focused ultrasound which over-heats samples in a manner similar to bead-beating. More importantly, this type of equipment costs $50,000-150,000 depending on throughput, making them inaccessible to smaller laboratories, particularly in resource-limited areas.

In addition to challenges associated with resilient microbes, low biomass pathogens are also hugely problematic, and researchers continually pursue methods to increase the extraction efficiency for these targets. High throughput streamlined methods to enrich target microbes and shear their DNA have the potential to accelerate patient diagnosis and improve microbial analysis for outbreak investigations. It would be enormously valuable to enrich microbes of a specific species or class before DNA extraction to substantially reduce noise, increase sequencing read depth, and provide improved starting samples for phenotypic (culturing-based) assays.

The present invention overcomes shortcomings in the art by providing a targeted particle that can be used to isolate and lyse a microbial cell of interest.

### SUMMARY OF THE INVENTION

The present invention provides cavitation-enhancing sonication and buoyant targeted particles for efficient, unbiased (both in terms of shearing of DNA and lysis of microbial populations), and reproducible enrichment and lysis of biological samples in low-cost, low-power bath sonicators and extraction of biomolecules. These particles enable low-pressure sonication of biological samples, thus reducing the acoustic energy required for cavitation and subsequently improving the precision of sonication-based lysis.

One aspect of the invention relates to a targeted particle, wherein the targeted particle comprises a shell, a core, and a targeting ligand, wherein the targeting ligand binds to the wall or membrane of a microbial cell.

A further aspect of the invention relates to a method for extracting biomolecules from a microbial cell, the method comprising: providing a particle which is a nanodroplet or a microbubble, the particle comprising a targeting ligand that binds the microbial cell, wherein the particle comprises a core (either gas or liquid) and a shell; producing a sonication sample by contacting a sample comprising the microbial cell with the particle, wherein the particle binds to the wall or membrane of the microbial cell; subjecting the sonication sample to a low-pressure sonication; and extracting biomolecules.

A further aspect of the invention relates to a method for selectively enriching a microbial cell of interest from a sample comprising the microbial cell, the method comprising: providing a particle, the particle comprising a targeting ligand that binds the microbial cell, the particle comprising a core and a shell; contacting the sample with the particle, wherein the particle binds the microbial cell; and floating the microbial cell bound to the particle to the top of the sample, producing an inverse pellet, thereby selectively enriching the microbial cell.

These and other aspects of the invention are set forth in more detail in the description of the invention below.

### BRIEF DESCRIPTION OF THE DRAWINGS

FIG. 1 illustrates traditional, phospholipid shelled nanodroplets. Nanodroplets exist in a metastable state where the perfluorocarbon core is in a superheated liquid phase (beyond its boiling point) until activation with ultrasound energy or increased temperature, at which point the superheated liquid converts into the gas phase. Additional ultrasonic energy can be used to drive the microbubble into an oscillatory state and eventually the microbubble collapses in an event known as inertial cavitation. During inertial cavitation, mechanical shear forces are generated with microstreaming and shockwaves. In this particular formulation, the shell comprises various phospholipids that affect stability and reduced coalescence; however, the shell can also be created using proteins, polymers, and other surfactants.

FIG. 2 illustrates targeted nanodroplets and microbubbles with molecular ligands. The shell composition of either version of the reagent comprises the same formulation methodology. The only formulation difference between nanodroplets and microbubbles is that the core of the nanodroplets is a superheated liquid state, while the core of the microbubbles is in a gaseous state. The targeting ligands can be any molecule, including but not limited to antibodies, lectins, and peptide sequences. The choice of ligand depends on the specific microbial target. For example, to specifically target mycobacteria, anti-lipoarabinomannan (LAM) antibody can be conjugated to the reagent shell. As another example, to generally target Gram-positive bacteria, Concanavalin A can be conjugated to the reagent shell. As a third example, to target Staphylococcus aureus, anti-Protein A can be conjugated to the reagent shell. The ligand can be conjugated to the shell using a number of chemistries depending on the shell composition. For example, in one instance when the shell of the reagent is a phospholipid, the ligand can be conjugated to a Dibenzocyclooctyne (DBCO) functionalized lipid using Click Chemistry where the ligand is reacted with an Azido-PEG-NHS ester molecular linker. The ligand can also be incorporated to the shell using other techniques including but not limited to thiol/maleimide and biotin/streptavidin chemistries.

FIG. 3 illustrates the differences between targeted nanodroplets (right) and targeted microbubbles (left). Targeted microbubbles have a gaseous core, are 1-3 microns in diameter, and are positively buoyant. Targeted nanodroplets have a liquid core, are 150-250 nm in diameter, and are neutrally buoyant. Nanodroplets are primarily used for cell lysis applications. The nanodroplet transition to a microbubble can be controlled in a fashion where inertial cavitation does not occur, and thus nanodroplets can be used as a precursor for cell enrichment applications. Microbubbles can be directly manufactured and are typically used for cell enrichment applications, although upon activation with ultrasound, the microbubbles can also inertially cavitate and be used for cell lysis if desired. Nanodroplets can be frozen or stored in a refrigerator for long-term storage without significant loss in quality. Microbubbles can also be stored in a refrigerator but can also be lyophilized for long-term storage.

FIG. 4 illustrates how the targeting ligands of the reagents preferentially bind to the target microbe.

FIG. 5 illustrates how buoyancy of targeted microbubbles can be used to separate or enrich target microbes out of suspension using centrifugation or naturally allowing the microbubbles to float to the surface. The separated microbubbles form a layer (often called a “bubble cake”) and can be separated from the infranatant or pellet. The cells collected in the bubble cake can then be lysed if desired, or cultured.

FIGS. 6A-6B illustrate targeted nanodroplets increase lysis extraction efficiency from resilient bacteria. (A) A comparison of DNA extraction efficiencies of traditional nanodroplets (Untarg), Concanavalin A targeted nanodroplets (ConA-SL), sonication alone (Veh), QIAamp BiOstic Bacteremia DNA kit (Q-Bacter; bead-beating), and DNeasy Blood and Tissue (Q-DN). For E. faecalis (1×10⁷ CFU/mL), targeted nanodroplets extract 4.8× more DNA than the bead-beating approach. For M. smegmatis (1×10⁶ CFU/mL), targeted nanodroplets extracts 6.5× and 122× more DNA than the bead-beating and chemical lysis approaches, respectively. (B) Gram-positive and negative bacteria and mycobacteria lysed using the same optimized conditions. DNA shearing is consistent between bacteria types suggesting low extraction and shearing bias.

FIGS. 7A-7D illustrate data demonstrating specificity of targeting ligand and cell recovery performance of targeted microbubbles. (A) FITC-labeled anti-Protein A antibody binding efficiency to S. aureus is significantly greater compared to a BSA control. (B) Specificity of FITC-labeled anti-Protein A antibody to S. aureus is significantly greater compared to E. coli and E. faecalis. (C) 71% recovery of S. aureus during microbial float assay using anti-Protein A antibody conjugated microbubbles, compared to the expected (calculated based on volume fraction of microbubble cake divided by infranatant). (D) 68% recovery of E. faecalis during microbial float assay using Concanavalin A conjugated microbubbles compared to a no-bubble control.

### DETAILED DESCRIPTION OF THE INVENTION

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

Except as otherwise indicated, standard methods known to those skilled in the art may be used for production of targeted particles, extracting biomolecules, sonicating, and centrifuging samples.

Moreover, the present invention also contemplates that in some embodiments of the invention, any feature or combination of features set forth herein can be excluded or omitted.

### Definitions

The following terms are used in the description herein and the appended claims.

The singular forms “a” and “an” are intended to include the plural forms as well unless the context clearly indicates otherwise.

Furthermore, the term “about,” as used herein when referring to a measurable value such as concentration, time, temperature, and the like, is meant to encompass variations of 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

The term “particle” as used herein refers to a particle that comprises a shell and a core, wherein the shell encompasses a liquid or gas at the core. A particle that is about 150-250 nm in diameter and encompasses a liquid at its core is referred to as a “nanodroplet”. A particle that is about 1-3 μm in diameter and encompasses a gas at its core is referred to as a “microbubble”.

The term “sonication” as used herein refers to a process wherein sound waves are used for dispersing particles in a sample, converting nanodroplets to microbubbles, and/or lysing a microbial cell.

The term “biomolecule” as used herein refers to any molecule that can be obtained from a cell, including without limitation, DNA, RNA, intracellular protein, intramembrane/cell wall protein, and metabolites. The biomolecule of the present invention can be from a microbial cell and/or a non-microbial cell, e.g., a host cell.

The term “downstream application” as used herein refers to any process that can used on the products produced by the methods of the invention, including without limitation, Next-Generation Sequencing, molecular diagnostics, quantitative polymerase chain reaction (qPCR) based diagnostics/analysis, enzymatic assays, and recombinant protein expression/production.

The term “bubble cake” refers to a layer of microbubbles formed during the enrichment methods of the invention and can be separated from the infranatant or pellet.

The term “infranatant” as used herein refers to a liquid lying below a bubble cake and or above a pellet.

The term “ligand” as used herein refers to any molecule or atom that binds to a receiving molecule, e.g., a protein molecule.

The term “enrichment” as used herein refers to increasing the concentration of a cell in a sample, e.g., by isolating or separating the cell from the sample or increasing the amount of the cell in the sample relative to other materials in the sample.

Infectious diseases are a leading cause of global morbidity and mortality, accounting for 29% of deaths worldwide. See., e.g., World Health Organization (WHO). The top 10 causes of death. In, 2018. Timely and accurate identification of pathogens facilitates efficient management of antimicrobial treatments, faster patient recovery, decreased use of broad-spectrum antibiotics (thereby inhibiting proliferation of drug-resistant variants), containment outbreaks, and reduction of medical costs. See., e.g., Chiang S S, Khan F A, Milstein M B, et al. Treatment outcomes of childhood tuberculous meningitis: a systematic review and meta-analysis. Lancet Infect Dis 2014; 14:947-57. Traditional microbiology techniques, including but not limited to microscopic examination, cell culture, and phenotypic drug sensitivity testing (pDST), are often considered the first-line of pathogen detection. However, these techniques are often inaccessible in resource-limited locations due to the need for dedicated laboratories, equipment, and training, and inherent technical limitations lead to inconclusive pathogen identification in up to 60% of cases. See., e.g., Colman R E, Suresh A, Dolinger D L, Muñoz T, Denkinger C M, Rodwell T C. Review of automated DNA extraction systems for sequencing-based solutions for drug-resistant tuberculosis detection. Diagn Microbiol Infect Dis. 2020; 98 (2):115096; Duan H, Li X, Mei A, et al. The diagnostic value of metagenomic next-generation sequencing in infectious diseases. BMC Infect Dis. 2021; 21 (1):62.

Molecular diagnostic tests, including qPCR-based Nucleic Acid Testing (NAT) and NGS, enable faster and more sensitive detection and characterization of pathogens with precise high-throughput technologies. See., e.g., Dulanto Chiang A, Dekker J P. From the pipeline to the bedside: advances and challenges in clinical metagenomics. The Journal of infectious diseases 2020; 221:S331-S40. Recent advances in sequencing techniques have poised NGS to be a future universal tool for infectious pathogen diagnostics, drug-susceptibility testing (DST), and strain identification, as researchers have successfully used NGS to identify genes leading to antimicrobial resistance. See., e.g., Chen H, Li J, Yan S, et al. Identification of pathogen(s) in infectious diseases using shotgun metagenomic sequencing and conventional culture: a comparative study. Peer J 2021; 9:e11699. This approach also allows researchers to perform real-time genomic epidemiology and drug resistance surveillance in areas without the facilities for cell culture and pDST. See., e.g., Goig G A, Cancino-Muñoz I, Torres-Puente M, et al. Whole-genome sequencing of Mycobacterium tuberculosis directly from clinical samples for high-resolution genomic epidemiology and drug resistance surveillance: an observational study. The Lancet Microbe 2020; 1:e175-e83. Whole genome sequencing (WGS) has become a key tool in public health surveillance, especially in pathogen diagnosis, tracking of disease outbreaks, and delineating the spread of antibiotic-resistant organisms. See., e.g., Revez J, Espinosa L, Albiger B, et al. Survey on the Use of Whole-Genome Sequencing for Infectious Diseases Surveillance: Rapid Expansion of European National Capacities, 2015-2016. Front Public Health 2017; 5:347. As early as mid-2016, half of countries in the European Union were using WGS analysis, either as first-or second-line methods, for surveillance of priority pathogens and tracking of antibiotic resistance. See., e.g., Revez J, Espinosa L, Albiger B, et al. Survey on the Use of Whole-Genome Sequencing for Infectious Diseases Surveillance: Rapid Expansion of European National Capacities, 2015-2016. Front Public Health 2017; 5:347. WGS has also become increasingly useful and affordable for such purposes: for example, WGS has been found to be 93% accurate in detecting and characterizing culture-enriched multidrug-resistant (MDR) M. tuberculosis (Mtb), with a 7% lower cost than phenotypic methods. See., e.g., Schürch A C, van Schaik W. Challenges and opportunities for whole-genome sequencing-based surveillance of antibiotic resistance. Annals of the New York Academy of Sciences 2017; 1388:108-20.

The use of WGS for diagnosis of bacterial and viral infections is more affordable than ever, and certain patients may benefit from such direct sample approaches. These patients may be immunocompromised due to cancer, have a hereditary condition, or have recently undergone transplantation, making them extremely vulnerable to both common and uncommon infections from viruses, bacteria, fungi, or parasites. In these cases, organism recovery from routine culture is often limited due to the use of broad-spectrum or prophylactic antimicrobial drugs or the presence of slow-growing or fastidious microbes, thereby making WGS analysis appealing. See., e.g., Gu W, Miller S, Chiu C Y. Clinical Metagenomic Next-Generation Sequencing for Pathogen Detection. Annu Rev Pathol 2019; 14:319-38. NGS-based techniques have been shown to have better diagnostic sensitivity than culture-based methods, with one study reporting a difference in sensitivity of 67.4% vs. 23.6% (P<0.001) between these methods, especially in sample types of bronchoalveolar lavage fluid (P=0.002), blood (P<0.001), and sputum (P=0.037). See., e.g., Duan H, Li X, Mei A, et al. The diagnostic value of metagenomic next-generation sequencing in infectious diseases. BMC Infectious Diseases 2021; 21:62.

Importantly, several countries have seen great success with implementing WGS for foodborne-pathogen surveillance. Worldwide, an estimated 1.9 billion people each year experience a food-borne infection, and 715,000 die from the infection, showing the great need for better surveillance. See., e.g., Organization WH. WHO estimates of the global burden of foodborne diseases: foodborne disease burden epidemiology reference group 2007-2015: World Health Organization; 2015. WGS for microbial pathogen surveillance has now been implemented in the US, UK, Denmark, and France. The year after WGS was implemented in the US for listeriosis surveillance, the number of outbreaks detected increased 36%, and the number of solved outbreaks increased more than three-fold. See., e.g., Besser J, Carleton H A, Gerner-Smidt P, Lindsey R L, Trees E. Next-generation sequencing technologies and their application to the study and control of bacterial infections. Clin Microbiol Infect 2018; 24:335-41. Additionally, smaller outbreaks were detected, outbreaks were detected earlier, and the total number of outbreak-related cases identified increased. See., e.g., Jackson B R, Tarr C, Strain E, et al. Implementation of Nationwide Real-time Whole-genome Sequencing to Enhance Listeriosis Outbreak Detection and Investigation. Clinical Infectious Diseases 2016; 63:380-6. The use of a WGS profile in outbreak investigations has also allowed cases to be ruled in or out of the outbreak with a higher degree of resolution than previously possible and can identify linked cases from a broader geographical and temporal range. See., e.g., Jagadeesan B, Gerner-Smidt P, Allard M W, et al. The use of next generation sequencing for improving food safety: Translation into practice. Food Microbiol 2019; 79:96-115. WGS of bacterial isolates has also been used successfully to track hospital infections, and outbreaks involving antibiotic-resistant bacteria. See., e.g., Besser J, Carleton H A, Gerner-Smidt P, Lindsey R L, Trees E. Next-generation sequencing technologies and their application to the study and control of bacterial infections. Clin Microbiol Infect 2018; 24:335-41.

Current Limitations of Sequencing: In pure culture, WGS is rapid and relatively inexpensive, and a powerful tool for genome characterization and identification of antibiotic resistance, among other applications. To cultivate sufficient microbial DNA for WGS, clinical samples must be cultured to increase sample load for extraction and isolate positively identified strains. However, culturing and isolating individual species can take days to weeks, and furthermore, many microbial species cannot be cultured with existing methods. Lastly, culturing also requires a complex laboratory infrastructure and equipment which is a considerable economic barrier in low resource regions, including low-and middle-income countries. See., e.g., Besser J, Carleton H A, Gerner-Smidt P, Lindsey R L, Trees E. Next-generation sequencing technologies and their application to the study and control of bacterial infections. Clin Microbiol Infect 2018; 24:335-41. Taken together, there is a global push in the infectious disease community for sequencing of culture-free samples to accelerate diagnosis and treatment.

Many clinical samples are made up of a mix of species (both microbial and host), and DNA extracted from these samples will be heavily contaminated with non-target species. For example, in human saliva, nasal cavity, skin, and vaginal samples, greater than 90% of the DNA is from the host. See., e.g., Marotz C A, Sanders J G, Zuniga C, DNA L S, Knight R, Zengler K. Improving saliva shotgun metagenomics by chemical host DNA depletion. Microbiome 2018; 6:42. In primary samples where the organisms of interest contribute a small percentage of nucleic acid to the total pool of extracted nucleic acid (as little as 0.002%), complete genomic resolution of a target microbe using shotgun sequencing is challenging and costly due to requirement for large quantities of starting material. Thus, successful downstream analysis of mixed samples depends on the amount of DNA from the organisms of interest relative to all other DNAs in the sample.

Targeted NGS (tNGS) enables rapid characterization and identification of pathogens where specific genes or gene regions are selectively enriched by PCR amplification to increase target signal to noise. This technique can be performed on direct clinical samples and is a viable alternative to WGS in developing countries that cannot afford dedicated cell culture laboratory facilities. Unfortunately, successful enrichment of the pathogen specific genes is highly dependent on sample extraction efficiency. As a result, culture-free tNGS of resilient pathogens (i.e., Gram-positive bacteria, mycobacteria, spores, etc.) can be problematic due to the impenetrability of the cell walls. According to the Center for Disease Control's report on Antibiotic Resistance Threats, over 66% of drug-resistant microbial species are gram-positive bacteria, mycobacteria, or difficult to lyse fungi. See, e.g., Antibiotic Resistance Threats in the United States, 2019. Atlanta, GA: U.S. Department of Health and Human Services, CDC; 2019. A major element in successfully implementing culture-free NGS for pathogen detection and DST is overcoming limitations in lysis and extraction of DNA from resilient microbes.

Current Challenges Associated with Microbial Lysis: To facilitate reliable clinical diagnostic workflows, there is need for high efficiency extraction of nucleic acids from hard-to-lyse microorganisms in direct patient samples. See., e.g., de Bruin O M, Birnboim H C. A method for assessing efficiency of bacterial cell disruption and DNA release. BMC microbiology 2016; 16:197. For example, bloodstream infections (BSIs) tend to be paucibacterial, and Gram-positive or fungal pathogens are found in roughly half of all patients. See., e.g., Naccache S N, Peggs K S, Mattes F M, et al. Diagnosis of neuroinvasive astrovirus infection in an immunocompromised adult with encephalitis by unbiased next-generation sequencing. Clin Infect Dis 2015; 60:919-23. Sputum is another clinical sample that is attractive for developers of diagnostics due to the opportunity for non-invasive sample collection. See., e.g., Leung E T, Zheng L, Wong R Y, et al. Rapid and simultaneous detection of Mycobacterium tuberculosis complex and Beijing/W genotype in sputum by an optimized DNA extraction protocol and a novel multiplex real-time PCR. J Clin Microbiol 2011; 49:2509-15; however, poor lysis efficiency drastically reduces the sensitivity and accuracy of NGS-approaches. For example, nontuberculous mycobacteria (NTM) respiratory infections in cystic fibrosis patients are underrepresented in diagnostic qPCR and 16s rDNA sequencing due to resilient cell walls that are difficult to rupture. A recent study showed that improvements in bacterial lysis efficacy led to improved NTM detection with both qPCR and 16s rDNA gene sequencing. See., e.g., Caverly L J, Carmody L A, Haig S-J, et al. Culture-Independent Identification of Nontuberculous Mycobacteria in Cystic Fibrosis Respiratory Samples. PLoS One 2016; 11:e0153876-e.

Many disease-causing Gram-positive bacteria, mycobacteria, and fungi are resistant to conventional cell lysis approaches and pose diagnostic challenges due to low bacterial loads. Resilient microbes are encapsulated in a cell wall containing peptidoglycans, lipoglycans, mycolic acids, and waxy layers that create an impenetrable barrier. Chemical and enzymatic methods have demonstrated some success with Gram-positive bacteria; however, disadvantages include poor lysis efficiency, difficulty with chemical and enzyme purification, sensitivity to buffers containing compounds such as EDTA, and complex protocols that preclude routine use in laboratory settings. Although these methods may be compatible with amplification-based techniques, they are not well suited for NGS as sequencing requires a greater input of DNA, which can only be achieved with greater lysis efficiency (or culturing).

Current Challenges Associated with Microbial Enrichment: Current approaches to isolate and characterize specific pathogens include the use of specialized media and specific culture conditions. However, these techniques are time-, labor-, and resource-intensive. See. e.g., Lecuit M, Eloit M. The diagnosis of infectious diseases by whole genome next generation sequencing: a new era is opening. Front Cell Infect Mi 2014; 4:25. Because of this, methods that target specific genomic sequences like PCR or nucleic acid sequence-based amplification (NASBA) are commonly used. While PCR is rapid, affordable, sensitive, and specific, it is only able to identify predefined regions of the genome. In addition, for pathogens that are highly variable like RNA or DNA viruses, PCR-based tests cannot discriminate between genotypes. See., e.g., Lecuit M, Eloit M. The diagnosis of infectious diseases by whole genome next generation sequencing: a new era is opening. Front Cell Infect Mi 2014; 4:25. While WGS overcomes these limitations, host cells/DNA can overpower the signal in clinical samples, significantly driving down sequencing depth, increasing costs, and making it difficult to assemble complete genomes for low biomass species.

There are a handful of existing approaches to remove host cells/DNA that can be applied before or after DNA extraction. Sample filtration can remove comparatively large mammalian cells, but one study of saliva found that substantial amounts of extracellular mammalian DNA persisted after filtration and continued to contaminate the sample. See., e.g., Marotz C A, Sanders J G, Zuniga C, DNA L S, Knight R, Zengler K. Improving saliva shotgun metagenomics by chemical host DNA depletion. Microbiome 2018; 6:42. Another pre-extraction method involves lysing mammalian cells and degrading exposed DNA. However, the multiple wash steps required can remove low-biomass species, and researchers have reported a potential bias toward Gram-positive organisms. See. e.g., Horz H P, Scheer S, Huenger F, Vianna M E, Conrads G. Selective isolation of bacterial DNA from human clinical specimens. J Microbiol Methods 2008; 72:98-102. Additionally, approaches that use propidium monoazide (PMA) to crosslink extracellular DNA and block amplification may not be suitable for all applications. See., e.g., Marotz C A, Sanders J G, Zuniga C, DNA L S, Knight R, Zengler K. Improving saliva shotgun metagenomics by chemical host DNA depletion. Microbiome 2018; 6:42. Alternatively, one post-extraction approach targets methylated nucleotides, but shows a bias against AT-rich genomes and would be unsuitable for eukaryotic targets with similar methylation patterns. See., e.g., Liu G, Weston C Q, Pham L K, et al. Epigenetic segregation of microbial genomes from complex samples using restriction endonucleases hpaii and McrB. PLoS One 2016; 11:e0146064; Marotz C A, Sanders J G, Zuniga C, DNA L S, Knight R, Zengler K. Improving saliva shotgun metagenomics by chemical host DNA depletion. Microbiome 2018; 6:42. For samples that contain off-target microbial populations in addition to host cell contamination, there are no methods to enrich or isolate the microbe of interest out of the sample, apart from culturing.

### Ultrasonically Activatable Reagents With Molecular Ligands for Microbial Lysis

To overcome the current limitations of microbial lysis, the present invention uses a nanodroplet-based, cavitation-enhancing sonication reagent for efficient, unbiased, and reproducible cell lysis of biological samples in low-cost, low-power bath sonicators. This reagent reduces the acoustic energy required for cavitation, which subsequently improves the precision of sonication-based lysis. This provides a consistent, reliable, and efficient process that minimizes thermal effects and reduces sample loss or degradation. Moreover, this reagent enables the use of inexpensive, off-the-shelf sonicators that are very accessible to laboratories around the world. These instruments cost between $400 and $10,000 (comparable to bead-beaters) and expand the opportunity for researchers and clinicians to take advantage of this technology. Finally, labs and researchers will be able to effectively lyse and extract DNA from difficult-to-lyse microbes, such as Mtb. This reagent technology will support improved sample processing and entry into tNGS and eventually WGS workflows by combining lysis and controlled DNA shearing to a range that suit these applications (300-3,000 bp). This will ultimately translate to reduced workflow, increased throughput, and improved experimental rigor and reproducibility.

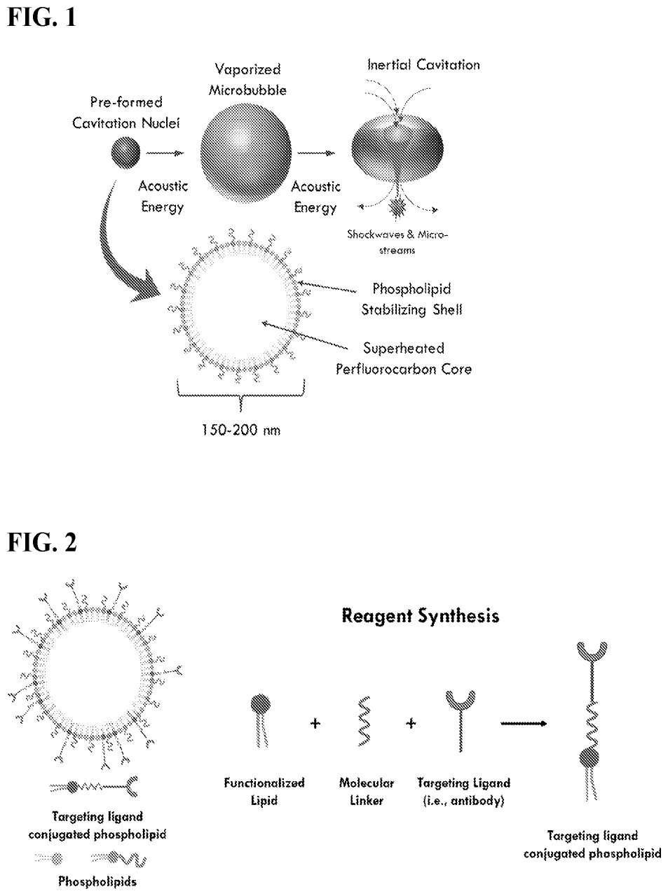

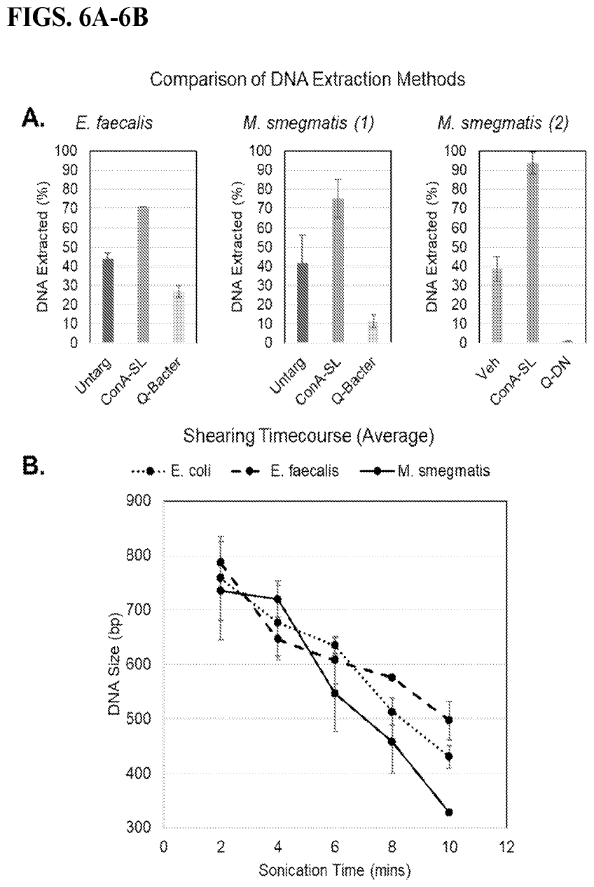

The newly conceived formulation of molecular-targeted nanodroplets is designed specifically for enhancing lysis efficiency of resilient microbes. With traditional nanodroplet-based sonication reagents (FIG. 1), the reagent is simply mixed into a sample containing the biological material. During sonication, the cavitation events occur probabilistically, and there is an inverse correlation between the distance from a biological material (i.e., cell) to a nanodroplet, and the force absorbed by the biological material (i.e., cells in close proximity to a nanodroplet, will experience greater shear forces). See., e.g., Meng et al., Adv Sci (Weinh) 6 (17):1900557 (2019); Wang et al., Sci Rep 8 (1):3885 (2018); Guo et al., Ultrason Sonochem 39:863 (2017); Ward et al., Ultrasound Med Biol 26 (7):1169 (2000). For resilient microbial samples, this inverse correlation is significant, as lysis efficiency rapidly decreases as the density of cavitation events is reduced. Recent data demonstrate a significant (up to 100×) improvement in DNA extraction from Mycobacterium smegmatis (a Mycobacterium used as a model for Mtb) and Enterococcus faecalis (a Gram-positive bacteria), respectively, compared to commonly used commercial kits (FIG. 6A).

The nanodroplet reagents may be composed of metastable perfluorocarbon nanodroplets See., U.S. Pat. No. 9,427,410. The nanodroplets may comprise a superheated perfluorocarbon gas (e.g., perfluorobutane, boiling point −2° C.) that has been condensed to a liquid form and stabilized by a phospholipid monolayer shell. The targeted formulation contains targeting ligands incorporated into the lipid shell, enabling direct binding to specific components on the target cell surface (FIG. 2). The nanodroplets range from 100-200 nanometers in diameter and remain metastable in solution until exposed to an acoustic field, at which point these liquid nanodroplets vaporize into gas-filled microbubbles. They subsequently collapse (inertial cavitation), providing explosive microbursts of mechanical energy in the form of shockwaves and microstreams. These nanodroplets are designed for incorporation into acoustic processing of biological samples. See., U.S. Pat. No. 9,982,290. The application-specific reagent can be simply added into the sample in a 1:10 v/v ratio (or other experimentally determined ratios) in order to substantially decrease the acoustic energy required for cavitation to occur and allow for more precise control over cavitation. The innovative aspects of this technology include the following. (A) Nanodroplets enable efficient use of ultrasound for the lysis of microbes. The cavitation enhancement provided by nanodroplets drastically improves the consistency and efficiency of sonication, reducing the total acoustic energy required to lyse microbial samples and, consequently, minimizing damage or over-fragmentation of nucleic acids. Additionally, by lowering the acoustic threshold for cavitation using nanodroplets, high-throughput multi-sample processing of up to 96 microbial samples is possible. Nanodroplet-enhanced sonication will potentially surpass the industry standard bead beating, as a safer, more streamlined, higher throughput process for lysis of resilient microbes. (B) Molecular-targeted nanodroplets further enhances lysis of microbial samples: Nanodroplet shell compositions can be easily tailored to bind to the cell walls of specific types of microbes. The present data and previous literature suggest that nanodroplet distance to the cell wall is inversely proportional to lysis efficiency; thus, nanodroplets that bind to select difficult-to-lyse microbes can deliver more focused cavitation. The implications of the targeted approach of the present invention include (1) more efficient lysis of samples with low bacterial loads, and (2) unbiased lysis of a mixed population using a cocktail of targeted nanodroplet formulations, thus focusing energy to a range of resilient microbes (Gram-positives, mycobacteria, fungi, spores, etc.). (C) Nanodroplets enable the use of low-power sonicator devices that can be miniaturized for point-of-care applications: The current industry-standard high-powered focused sonicators deliver large amounts of energy and heat into the sample, resulting in sample degradation. Nanodroplets require substantially less acoustic energy to achieve the same level of cavitation within a sample. As a result, low power sonicators (that can be easily designed for microfluidic type devices in point-of-care settings) may be used in the future.

### Targeted Nanodroplet and Microbubble Compositions

In one aspect, the present invention provides targeted particles for lysis of a microbial cell of interest and extraction of biomolecules from the microbial cell. Particles can be microbubbles or nanodroplets. The shell of the targeted particles is conjugated to a targeting ligand (e.g., a lectin, antibody, and/or peptide) that binds the membrane or cell wall of a microbial species of interest. This closes the gap between the inertial cavitation event and subsequent shearing forces and the cell, thus maximizing the transfer of energy and lysis potential. Once sonicated, nanodroplets vaporize into microbubbles. Microbubbles or resultant microbubbles from nanodroplet vaporization oscillate in the presence of an acoustic field and generate localized shear force. Microbubbles may oscillate continuously in as stable manner (stable cavitation) without complete dissolution or rupture of the shell. The microbubble may violently oscillate and implode (inertial cavitation) characterized by complete rupture of the shell.

In another aspect, the present invention provides targeted particles for enrichment of a microbial cell of interest. Microbubbles or resultant microbubbles from vaporization of nanodroplets may be used for enrichment of a microbial cell of interest from a sample. Microbubbles bind the membrane or cell wall of a microbial species of interest and make the cells float on top of a suspension.

In some embodiments, the targeted particle comprises a shell, a core, and a targeting ligand, wherein the targeting ligand binds to the wall or membrane of a microbial cell. The targeting ligand may bind to a protein, glycoprotein, sugar, lipid, or other molecule present in the wall or membrane.

In some embodiments, the targeting ligand of the targeted is a lectin, antibody, or peptide.

In some embodiments, the targeting ligand of the particle is a lectin, e.g., wherein the lectin is concanavalin A (targeting Gram-positive bacteria), wheat germ agglutinin, dectin-1 (targeting Candida), or mannose/mannan binding protein (targeting Candida).

In some embodiments, the targeting ligand of the particle is an antibody, e.g., wherein the antibody is an anti-lipoarabinomannan antibody, an anti-protein A antibody (targeting Staphylococcus), a lipoteichoic acid antibody, an anti GM 1 antibody, a Candida albicans antibody, a Neisseria antibody (e.g., an anti-Neisseria MOMP antibody), a Mycobacterium antibody (e.g., an anti-Mycobacteria LAM antibody), or an Enterococcus antibody.

In some embodiments, the targeting ligand of the particle is conjugated to the shell of the particle.

In some embodiments, the targeting ligand of the particle is conjugated to the shell by click chemistry, thiol/maleimide, thiol/thiol, hydrazide/aldehyde, gold/thiol, or biotin/streptavidin-based techniques.

In some embodiments, the shell of the particle comprises a surfactant (e.g., a lipid), a protein, and/or a polymer.

In some embodiments, the shell of the particle comprises 1,2-distearoyl-sn-glycero-3-phosphoethanolamine (DSPE), polyethylene glycol (PEG), 1,2-distearoyl-sn-glycero-3-phosphocholine (DSPC), 1,2-dipalmitoyl-sn-glycero-3-phosphocholine (DPPC), dibenzocyclooctyne (DBCO), 1,2-distearoyl-sn-glycero-3-phosphoethanolamine-N-[methoxy (polyethylene glycol)-2000] (DSPE-PEG2000), another DSPE with another PEG chain length, and/or DBCO with a click chemistry linker.

In some embodiments, the targeting ligand of the particle ligand is concanavalin A, wherein concanavalin A is conjugated to the shell with an azide conjugate.

In some embodiments, the particle comprises a liquid or gaseous core.

In some embodiments, the core of the particle comprises a gas with a boiling point of −100° C. to 50° C., e.g., −75° C. to 50° C. or −50° C. to 50° C.

In some embodiments, the core of the particle comprises a perfluorocarbon.

In some embodiments, the core of the particle comprises a perfluorocarbon, wherein the perfluorocarbon is octofluoropropane, decafluorobutane or hexafluoropentane.

In some embodiments, the core of the particle comprises sulfur hexafluoride.

### Method for Lysis and Extraction of Biomolecules From a Microbial Cell

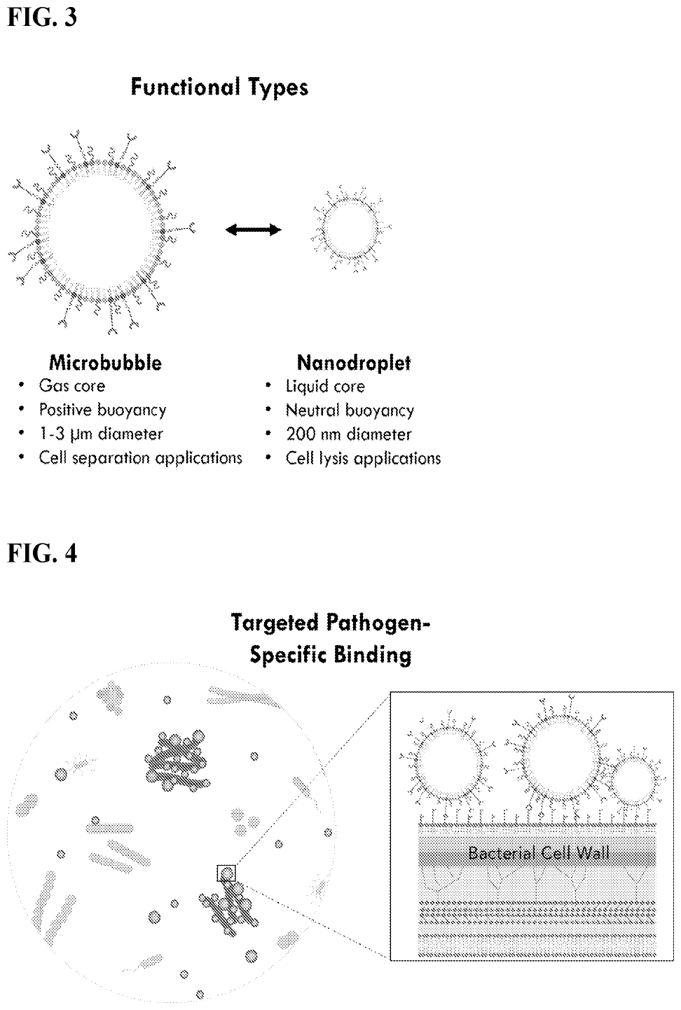

The present invention further provides methods for lysing a microbial cell and extracting biomolecules from the microbial cell. The method involves the chemical conjugation of targeting ligands to the shell of ultrasonically activatable nanodroplets (targeted nanodroplets) and microbubbles (targeted microbubbles) (FIG. 3). The targeting ligands are determined according to the application (e.g., lysis and/or enrichment of Gram-positive bacteria, lysis and/or enrichment of specific microbial species such as Mycobacterium tuberculosis, etc.). For lysis applications, the targeted nanodroplets and/or targeted microbubbles are introduced to any number of microbes contained in a sample chamber and allowed to bind to the cell wall or membrane of the target microbe (FIG. 4). The microbial sample is subsequently exposed to specific ultrasonic conditions which cause the nanodroplets or microbubbles to violently oscillate and eventually inertially cavitate, releasing localized mechanical shear forces in the form of micro-streaming and shockwaves. This addition of microscopic mechanical shear forces significantly enhances lysis efficiency of all microbes.

In some embodiments, the present invention provides a method for extracting biomolecules from a microbial cell, the method comprising: providing a particle which is a nanodroplet or a microbubble, the particle comprising a targeting ligand that binds the microbial cell, wherein the particle comprises a core and a shell; producing a sonication sample by contacting a sample comprising the microbial cell with the particle, wherein the particle binds to the wall or membrane of the microbial cell; subjecting the sonication sample to a low-pressure sonication; and extracting biomolecules.

In some embodiments, extraction of biomolecules is carried out at a frequency of about 1 kHz-100 MHz, e.g., about 10 kHz-50 MHz, e.g., about 20 kHz-10 MHz, e.g., about 20 kHz-2 MHz.

In some embodiments, extraction of biomolecules is carried out at a ratio of the particle to the microbial sample of about 1:50 v/v to about 1:1 v/v, e.g., about 1:20 v/v to about 1:5 v/v, e.g., about 1:10 v/v.

In some embodiments, the concentration of the particles in the sample is in the range of about 1×10² to about 1×10¹² particles per ml or any range therein.

In some embodiments, extraction of biomolecules is carried out on an environmental, clinical, or culture sample. Environmental samples may include, for example, water, soil, or surfaces. Clinical samples may include any biological sample that may contain microbes, e.g., from a subject suspected of having a microbial infection. The biological sample may be, for example, blood, serum, plasma, urine, saliva, semen, prostatic fluid, nipple aspirate, lachrymal fluid, perspiration, feces, cheek swabs, cerebrospinal fluid, cell lysate samples, amniotic fluid, gastrointestinal fluid, biopsy tissue, lymphatic fluid, or cerebrospinal fluid. Culture samples include, for example, environmental or clinical samples that have been cultures to grow any microbes that may be present. The sample may be, e.g., fresh, frozen, or fixed (e.g., with formalin).

The microbe may be any microbe that can be detected, separated, and/or lysed by the methods of the invention. Microbes include, without limitation, Gram-positive and negative bacteria, fungi, spores, viruses, phage, parasites, prions, etc. Bacteria include, without limitation, Candida, Neisseria, Mycobacterium, Staphylococcus, and Enterococcus species. The term “microbe” or “microbial cell” as used herein refers to a microbe, collection of microbes, biofilm, or a combination thereof.

### Buoyant Reagents With Molecular Ligands for Microbial Enrichment

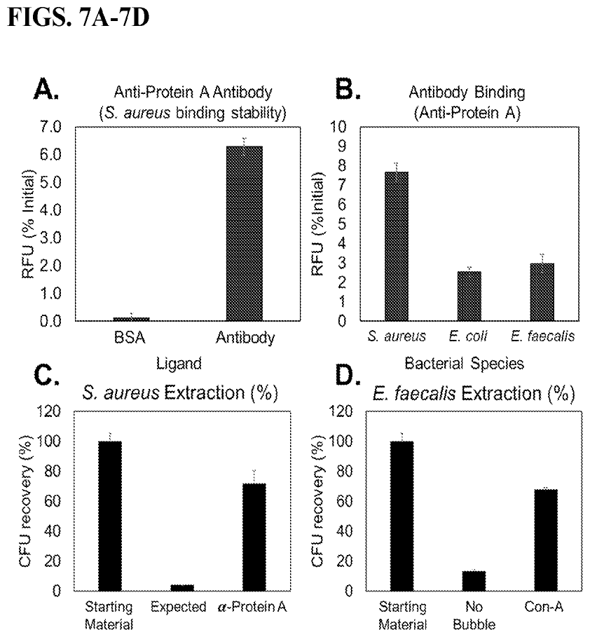

Similar to the targeted nanodroplets, the targeted microbubbles may comprise a monolayer phospholipid shell with a perfluorocarbon core. In contrast to the nanodroplets, the perfluorocarbon is in a gaseous phase (instead of a superheated liquid phase). Also, in contrast to the nanodroplets which are neutrally buoyant and will remain in suspension (or pellet with high-speed centrifugation), the microbubbles are positively buoyant and will eventually float out of solution. The targeted microbubble formulation contains ligands conjugated to the lipid shell, enabling direct binding of components to target cell surfaces. These ligands can be, without limitation, antibodies, unique peptide sequences, lectins, or other proteins that are specific for a target microbe (or class of microbes). For example, anti-Protein A antibody can be incorporated into the microbubble lipid membrane to target the surface of Staphylococcus aureus (FIGS. 7A-7D).

These microbubbles (1-3 μm in diameter) remain stable in solution and can be directly added to a sample, e.g., in a 1:10 v/v ratio, and then gently tumbled, e.g., at 4°° C. for 30-240 minutes. The samples subsequently undergo centrifugation, where bacteria bound by the targeted microbubbles float to the top of the sample vessel. The remaining sample is pelleted or remains in suspension, allowing for easy removal. Upon separating the microbubble targeted cell pellet or “cake” from the rest of the sample, the microbubble-bound cell mixture can be resuspended and sonicated for cell lysis and DNA extraction, or the sample can be used directly for cell culturing if desired. The innovative aspects of the microbubble technology include the following. (A) Pathogen specific targeting: The targeted microbubble is a platform technology that allows the development of microbubble formulations for any microbe of interest (Gram-positive and negative bacteria, fungi, spores, viruses, etc.). The ligand conjugation methodologies are being developed to be agnostic of the specific ligand and therefore can be designed for any microbial enrichment application. In this way, custom microbubble design is easy and economical. (B) Simultaneous microbial cell enrichment and lysis: The targeted microbubble acts as an enrichment reagent, as well as a lysis reagent. By simply sonicating the sample, the microbubble inertially cavitates (a violent collapse of the microbubble), releasing localized mechanical shear forces that efficiently rupture the cell wall of the microbe. The inventors' previous data demonstrate a >100× increase in the lysis efficiency of Mycobacteria using these lysis reagents, compared to commercially available bead-beating methods. By optimizing the sonication time, the level of DNA shearing can be controlled as well (from 200 to 3,000 bp) (FIG. 6B). The dual function of targeted microbubbles provides a streamlined sample processing solution for those interested in direct entry into NGS library preparation. (C) Accessibility and ease-of-use: The targeted microbubble workflow can be performed with simple, off the shelf plastic consumables, such as non-needle plastic syringes and a clinical centrifuge. Additionally, the microbubbles can be lyophilized for economical sale and distribution of the reagent, as well as efficient storage and increased shelf-life. Combining these two factors provides an easy-to-use solution that requires minimal laboratory infrastructure, ideal for low-resources settings such as low-and middle-income countries.

Another unique benefit of the microbubbles is that they can easily be disintegrated after enrichment if the downstream assay is culturing or some assay that requires the cells to be un-lysed/alive. Instead of having to use enzymes or competitive biotinylated compounds to release the bubble, a low-amount of negative pressure (innocuous to the cells) will disintegrate the bubbles.

The targeted microbubble technology of the present invention offers a way to isolate microbes of interest from clinical or environmental samples. Alternatively, the workflow could be used to remove host cells instead, to enrich for the microbial community as a whole. This technology can rapidly concentrate cells of interest for WGS, and provide enriched microbial communities for metagenome sequencing efforts. The approach can make NGS-based applications more accessible for microbial pathogen diagnostics, surveillance, and research.

The microbubble platform will allow technicians and researchers to bypass culturing steps to yield enriched populations of microbial species for molecular analysis. Alternatively, the platform could be used to isolate target microbes for further culture and phenotypic studies. As a result, phenotypic culturing-based studies can be started simultaneously alongside qPCR (or dPCR) diagnostic workflows and full NGS-based molecular characterization of target pathogens.

A critical factor for the routine use of NGS technologies in clinical microbiology is automation, and the targeted microbubble platform can be outfitted for rapid sample processing. This option offers additional time savings by enabling targeted isolation and concentration from clinical samples, coupled with microbial lysis and DNA shearing for downstream analysis through the cavitation-enhancing microbubbles.

Use of microbubble-conjugated ligands will also have diverse research applications in microbiology and other fields. It can be used to isolate subsets of the gut microbiome with specific functions in the body, or to study currently unculturable microbes. An added benefit of the microbubble approach lies in the ability to start with large sample volumes and concentrate the target microbes into smaller working volumes. This allows end users to harvest enough material from an extremely dilute sample for entry into downstream molecular applications.

Finally, the platform will not just enrich microbial targets of interest, but may have applications to enrich viruses or phages—such as to recover intact COVID viral particles from wastewater—or to isolate tumor cells from biopsy or blood samples. The strength of the technology lies in its flexibility for customization, where combinations of ligands can be used to pull out multiple classes of microbes, including bacteria, viruses, phage, parasites, and fungi.

### Method for Enhanced Microbial Enrichment

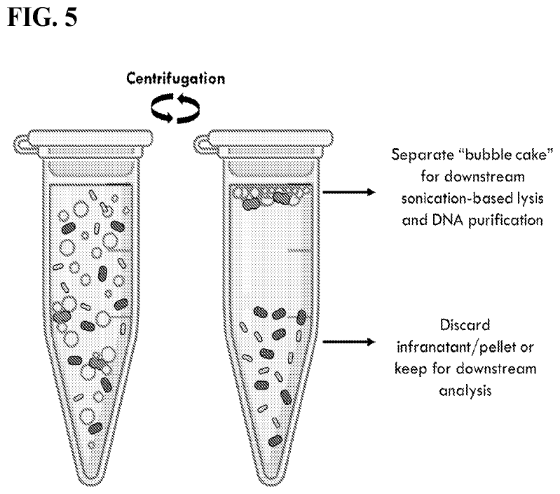

The present invention further provides methods for enrichment of a microbial cell. For enrichment applications, the targeted nanodroplets and/or microbubbles are introduced to a sample of microbes as previously described and allowed to bind to the cell wall or membrane of the target microbe. If nanodroplets are used, they can first be ultrasonically activated into microbubble form, otherwise the sample is allowed to sit or is centrifuged and the microbubble-bound microbes float to the top of the vessel while the rest of the sample suspension either remains in suspension or sinks to form a pellet (FIG. 5). The infranatant is removed and the microbubble layer containing the target microbes is eluted into a smaller volume.

In some embodiments, the present invention provides a method for selectively enriching a microbial cell of interest from a sample comprising the microbial cell, the method comprising: providing a particle, the particle comprising a targeting ligand that binds the microbial cell, the particle comprising a core and a shell; contacting the sample with the particle, wherein the particle binds the microbial cell; and floating the microbial cell bound to the particle to the top of the sample, producing an inverse pellet or bubble cake, thereby selectively enriching the microbial cell.

In one particular embodiment, the present invention provides a method for selectively enriching a microbial cell of interest from a sample comprising the microbial cell, the method comprising: providing a particle which is a microbubble, the particle comprising a targeting ligand that binds the microbial cell, the particle comprising a core and a shell; contacting the sample with the particle, wherein the particle binds the microbial cell; and floating the microbial cell bound to the particle to the top of the sample, producing an inverse pellet, thereby selectively enriching the microbial cell.

In another particular embodiment, the present invention provides a method for selectively enriching a microbial cell of interest from a sample comprising the microbial cell, the method comprising: providing a particle which is a nanodroplet, the particle comprising a targeting ligand that binds the microbial cell, the particle comprising a core and a shell; contacting the sample with the particle, wherein the particle binds the microbial cell; and floating the microbial cell bound to the particle to the top of the sample, producing an inverse pellet, thereby selectively enriching the microbial cell, and the method further comprises sonicating the sample after the contacting step and before the floating step, wherein the sonicating does not produce inertial cavitation, wherein the particle vaporizes into a microbubble.

The liquid core (droplet) can be used as a precursor to the bubble, meaning liquid core particles can bind to the cell of interest, and then the particle core can undergo a phase shift from liquid to gas under specific conditions (e.g., ultrasonic energy, increased heat, reduced pressure, etc.), forming a bubble. The conditions must be such that the bubble that forms does not cavitate (burst), so that it can function as a floatation device. Using nanodroplets as a precursor may be helpful in instances where long incubation times are necessary to bind to the cells. Nanodroplets are more stable and are less likely to lose function then microbubbles. Once the incubation time is over, the nanodroplets can be vaporized into microbubbles, and then the cells can be enriched.

Enrichment can be positive enrichment by targeting and separating the cell of interest or negative enrichment by targeting and removing other materials (e.g., host cells, non-target microbes). Positive selection may be used when there is a specific target microbe or other cell of interest. Negative enrichment may be useful, e.g., for microbiome studies where access to all microbes in the sample is desired and host cells are depleted.

In some embodiments, the methods can be used for enrichment of non-microbial cells. The cells may be, without limitation, mammalian or plant cells.

In some embodiments, the floating is performed by centrifuging the sample.

In some embodiments, the floating is performed naturally (e.g., the microbial cell bound to the particle is allowed to float to the surface on its own) without any additional steps.

In some embodiment, the centrifuging is performed in a syringe, plugged/capped pipette tip, capillary tube (e.g., with scored regions to separate the microbubble cake from the infranatant), or centrifuge tube.

In some embodiments, the enriched microbial cells are resuspended.

In some embodiments, the method further comprises extracting biomolecules from the enriched microbial cell, e.g., using the methods of the invention described above.

Having described the present invention, the same will be explained in greater detail in the following examples, which are included herein for illustration purposes only, and which are not intended to be limiting to the invention.

### EXAMPLES

Different extraction techniques were tested to compare DNA extraction efficiencies (FIGS. 6A-6B). Traditional nanodroplets (Untarg), Concanavalin A targeted nanodroplets (ConA-SL), sonication alone (Veh), QIAamp BiOstic Bacteremia DNA kit (Q-Bacter; bead-beating), and DNeasy Blood and Tissue (Q-DN) were tested. For E. faecalis (1×10⁷ CFU/mL), targeted nanodroplets extract 4.8× more DNA than the bead-beating approach. For M. smegmatis (1×10⁶ CFU/mL), targeted nanodroplets extracts 6.5× and 122× more DNA than the bead-beating and chemical lysis approaches, respectively. (B) Gram-positive and negative bacteria and mycobacteria were lysed using the same optimized conditions. DNA shearing is consistent between bacteria types suggesting low extraction and shearing bias. The data show that targeted nanodroplets increase lysis extraction efficiency from resilient bacteria.

To validate the quality and yield of extracted DNA, 16s rDNA qPCR was performed and the amplification efficiency compared between targeted nanodroplet enhanced sonication and conventional bead-beating based commercially available methods. Using the optimized conditions previously described, E. faecalis samples were lysed using both the targeted nanodroplet workflow and Qiagen Bacteremia kit, at cell concentrations ranging from 1×10¹-1×10⁷ CFU/mL, and a sonication time of 6 minutes. Biological and technical triplicates were included for each condition. For 16s rDNA gene qPCR, bacterial DNA concentrations were measured using a qPCR assay that targets the V3-V4 region of the 16S gene (90 bp amplicon). Escherichia coli 16S rDNA gene plasmid standards were run for each reaction ranging from 1×107 to 1×1010 copies/μL. The primary metrics were the Ct (doubling cycles) value and gene copies/μL. The results demonstrated up to 4.9× (ΔCt=−1.85) and 23.9× (ΔCt=−5.12) increase in copies/μL (decrease in Ct) after qPCR, comparing negative controls (vehicle; sonication only) and the Qiagen Bacteremia kit. Using targeted nanodroplets, 16S copies were detected at cell concentrations as low as 1×10³ CFU/mL, whereas the Bacteremia kit had a lower limit of detection of 1×10⁴ CFU/mL.

TABLE 1
Table 1. qPCR Performance of SnapLyse
over Vehicle and Qiagen Bacteremia Kit

| Bacterial | | | Fold Improvement In | |
|---|---|---|---|---|
| Concentration | ΔCt | | 16S Copies Detected | |
| (E. Faecalis; | | Qiagen | | Qiagen |
| CFU/mL) | Vehicle | Bacteremia | Vehicle | Bacteremia |
| 1.00E+07 | −1.18 | −4.65 | 1.12 | 19.65 |
| 1.00E+06 | −1.69 | −5.12 | 3.95 | 23.85 |
| 1.00E+05 | −1.85 | −4.34 | 4.93 | 10.68 |
| 1.00E+04 | −1.08 | −3.72 | 2.61 | 5.08 |

Further studies validate suitability for downstream qPCR amplification and demonstrated up to ˜24× increase in copies/μL, and a 1 order of magnitude improvement in the limit of detection (1×10³ CFU/mL), compared to commercial kits. These promising positive results suggest a significant impact on a wide range of applications requiring reliable microbial lysis techniques, including but not limited to, clinical and environmental microbiome studies where resilient microbes can be underrepresented in metagenomic analysis, NGS based food safety testing for infectious pathogens, and NGS for infectious disease detection and diagnosis.

The specificity of targeting ligand and cell recovery performance of targeted microbubbles was tested (FIGS. 7A-7D). The results showed that FITC-labeled anti-Protein A antibody binding efficiency to S. aureus is significantly greater compared to a BSA control (FIG. 7A). The specificity of FITC-labeled anti-Protein A antibody to S. aureus was significantly greater compared to E. coli and E. faecalis (FIG. 7B). A 71% recovery of S. aureus was seen during a microbial float assay using anti-Protein A antibody conjugated microbubbles, compared to the expected (calculated based on volume fraction of microbubble cake divided by infranatant) (FIG. 7C). A 68% recovery of E. faecalis during microbial float assay using Concanavalin A conjugated microbubbles, compared to a no-bubble control (FIG. 7D).

The foregoing examples are illustrative of the present invention, and are not to be construed as limiting thereof. Although the invention has been described in detail with reference to preferred embodiments, variations and modifications exist within the scope and spirit of the invention as described and defined in the following claims.

1. A method for extracting biomolecules from a microbial cell, the method comprising;
providing a particle which is a nanodroplet or a microbubble, the particle comprising a targeting ligand that binds the microbial cell, wherein the particle comprises a core and a shell;
producing a sonication sample by contacting a sample comprising the microbial cell with the particle, wherein the particle binds to the wall or membrane of the microbial cell;
subjecting the sonication sample to a low-pressure sonication; and
extracting biomolecules.

2. The method of claim 1, wherein the targeting ligand is a lectin (e.g., concanavalin A, wheat germ agglutinin, or mannose/mannan binding protein), an antibody (e.g., an anti-lipoarabinomannan antibody, an anti-protein A antibody, a lipoteichoic acid antibody, or an anti GM 1 antibody), or a peptide.

3-4. (canceled)

5. The method of claim 1, wherein the targeting ligand is conjugated to the shell of the particle (e.g., by click chemistry, thiol/maleimide, thiol/thiol, hydrazide/aldehyde, gold/thiol, or biotin/streptavidin-based techniques).

6. (canceled)

7. The method of claim 1, wherein the shell comprises a surfactant (e.g., a lipid), a protein, a polymer, or a combination thereof (e.g. wherein the shell comprises 1,2-distearoyl-sn-glycero-3-phosphoethanolamine (DSPE), polyethylene glycol (PEG), 1,2-distearoyl-sn-glycero-3-phosphocholine (DSPC), 1,2-dipalmitoyl-sn-glycero-3-phosphocholine (DPPC), dibenzocyclooctyne (DBCO), 1,2-distearoyl-sn-glycero-3-phosphoethanolamine-N-[methoxy (polyethylene glycol)-2000] (DSPE-PEG2000), another DSPE with another PEG chain length, and/or DBCO with a click chemistry linker).

8. (canceled)

9. The method of claim 1, wherein the particle comprises a liquid or gaseous core, e.g., wherein the core comprises a gas with a boiling point of −100°° C. to 50°° C., e.g., wherein the core comprises a perfluorocarbon, e.g., wherein the perfluorocarbon is octofluoropropane, decafluorobutane, or hexafluoropentane, e.g., wherein the core comprises sulfur hexafluoride.

10-13. (canceled)

14. The method of claim 1, wherein the sonication is carried out at a frequency of about 1 kHz-100 MHz.

15. The method of claim 1, wherein the ratio of the particle to the sample is 1:10 v/v.

16. The method of claim 1, wherein the concentration of the particles in the sonication sample is about 1×10² to about 1×10¹² particles per ml.

17. The method of claim 1, wherein the sample is an environmental, clinical, or culture sample.

18. The method of claim 1, wherein the microbial cell is a Gram-positive or negative bacterium, fungus, spore, virus, phage, parasite, or prion.

19. The method of claim 1, wherein the microbial cell is a microbe, collection of microbes, biofilm, or a combination thereof.

20. (canceled)

21. A method for selectively enriching a microbial cell of interest from a sample comprising the microbial cell, the method comprising:
providing a particle which is a nanodroplet or microbubble, the particle comprising a targeting ligand that binds the microbial cell, the particle comprising a core and a shell;
contacting the sample with the particle, wherein the particle binds the microbial cell; and
floating the microbial cell bound to the particle to the top of the sample, producing an inverse pellet, thereby selectively enriching the microbial cell.

22. (canceled)

23. The method of claim 21, wherein the particle is a nanodroplet and the method further comprises sonicating the sample after the contacting step and before the floating step, wherein the sonicating does not produce inertial cavitation, wherein the particle vaporizes into a microbubble, e.g., wherein the floating is performed naturally without any additional steps or wherein the floating is performed by centrifuging the sample, e.g., wherein the centrifuging is performed in a syringe, plugged/capped pipette tip, capillary tube, or centrifuge tube.

24-25. (canceled)

27. The method of claim 21, wherein the enriched microbial cells are resuspended.

28. The method of claim 21, further comprising extracting biomolecules from the enriched microbial cell.

29-48. (canceled)

49. A nanodroplet of microbubble particle, comprising:
a core;
a shell; and
a targeting ligand;
wherein the targeting ligand binds the wall or membrane of a microbial cell.

50. The particle of claim 49, wherein the targeting ligand is a lectin (e.g., concanavalin A, wheat germ agglutinin, or mannose/mannan binding protein), an antibody (e.g., an anti-lipoarabinomannan antibody, an anti-protein A antibody, a lipoteichoic acid antibody, or an anti GM 1 antibody), or peptide.

51-52. (canceled)

53. The particle of claim 49, wherein the targeting ligand is conjugated to the shell of the particle (e.g., by click chemistry, thiol/maleimide, thiol/thiol, hydrazide/aldehyde, gold/thiol, or biotin/streptavidin-based techniques)

54. (canceled)

55. The particle of claim 49, wherein the shell comprises a surfactant (e.g., a lipid), a protein, and/or a polymer (e.g., wherein the shell comprises 1,2-distearoyl-sn-glycero-3-phosphoethanolamine (DSPE), polyethylene glycol (PEG), 1,2-distearoyl-sn-glycero-3-phosphocholine (DSPC), 1,2-dipalmitoyl-sn-glycero-3-phosphocholine (DPPC), dibenzocyclooctyne (DBCO), 1,2-distearoyl-sn-glycero-3-phosphoethanolamine-N-[methoxy(polyethylene glycol)-2000](DSPE-PEG2000), another DSPE with another PEG chain length, and/or DBCO with a click chemistry linker).

56. (canceled)

57. The particle of claim 49, wherein the particle comprises a liquid or gaseous core, e.g., wherein the core comprises a gas with a boiling point of −100° C. to 50° C., e.g., wherein the core comprises a perfluorocarbon, e.g., wherein the perfluorocarbon is octofluoropropane, decafluorobutane, or hexafluoropentane, e.g., wherein the core comprises sulfur hexafluoride.

58-61. (canceled)
